(19) United States
(12) Patent Application Publication
(10) Pub. No.: US 2022/0002408 A1
(43) Pub. Date: Jan. 6, 2022

(54) BISPECIFIC ANTIBODY, PREPARATION METHOD THEREOF AND APPLICATION THEREOF

(71) Applicant: EXCYTELLC, Beijing (CN)

(72) Inventors: Andy Qingan YUAN, Beijing (CN); Qingwu MENG, Beijing (CN); Lili BAI, Beijing (CN); Likun ZHAO, Beijing (CN); Yanhu LI, Beijing (CN)

(21) Appl. No.: 17/290,957

(22) PCT Filed: Feb. 25, 2020
(86) PCT No.: PCT/CN2020/076516

§ 371 Date: May 3, 2021

(51) Int. Cl. C07K 16/28; C07K 16/46; A61P 35/00

(52) U.S. Cl. CPC C07K 16/2809; C07K 16/468; C07K 2317/73; C07K 2317/31; C07K 2317/622; A61P 35/00

(57) ABSTRACT

A bispecific antibody, a preparation method therefor and an application thereof. The bispecific antibody includes a monoclonal antibody unit and a single-chain antibody unit. The single-chain antibody unit includes two complete light chain-heavy chain pairs, and is specifically bound to a surface antigen of a tumor cell. The single-chain antibody unit includes two single-chain antibodies. The single-chain antibody includes a heavy chain variable region and a light chain variable region, and is specifically bound to a surface antigen of an immunocyte. The bispecific antibody can be simultaneously bound to the immunocyte and the tumor cell, can mediate a directed immune response, and can effectively kill the tumor cell.

### CROSS REFERENCE

The present application claims priority to Chinese patent application No. 201910209330.4 entitled “Bispecific antibody, preparation method thereof and application thereof”, filed on Mar. 19, 2019, the entire disclosure of which is incorporated herein by reference in its entirety.

### TECHNICAL FIELD

The present invention relates to the technical fields of biotechnology and immunology, specifically, to a bispecific antibody that binds CD19 and CD3, and preparation method thereof and use thereof.

### BACKGROUND ART

Antibody drugs are biomacromolecule drugs prepared by antibody engineering technologies with cell engineering technology and genetic engineering technology as the main body, and have the advantages of high specificity, uniform properties, and customized preparation for specific targets. Monoclonal antibodies are mainly used clinically in the following three aspects: tumor treatment, immune disease treatment and anti-infection treatment, wherein, tumor treatment is currently the most widely used field of monoclonal antibodies. Currently, among the current monoclonal antibody products that have entered clinical trials and are on the market, products used for tumor treatment account for approximately 50%. Monoclonal antibody treatment of tumors is an immunotherapy aimed at specific targets of diseased cells to stimulate the immune system to kill target cells. To enhance the effector function of antibodies, especially the effect of killing tumor cells, people have tried a variety of methods to modify antibody molecules. A bispecific antibody is one of the development directions to improve the therapeutic effect of antibodies and has become a hot spot in the field of antibody engineering research.

A bispecific antibody (BsAb) is an artificial antibody that can specifically recognize and bind to two different antigens or epitopes. If the two antigens are located on surface of different cells, the bispecific antibody can set up a bridge between the two antigen molecules, thereby forming cross-links between cells and mediating the cells to produce directed effector functions. BsAb has broad application prospects in biomedicine, especially in tumor immunotherapy. Bispecific antibodies (immune double antibodies) used for immunotherapy are artificial antibodies containing two specific antigen binding sites that bind to cell receptor antigens, and they can set up a bridge between diseased cells (target cells) and functional cells (immune cells), to stimulate a directed immune response. The killing of tumor cells by BsAb-mediated immune cells (such as T cells, NK cells and the like) is currently a hot spot in the application research of immunotherapy. The mechanism of action is that BsAb can simultaneously bind to tumor-related antigens and target molecules on immune effector cells, and directly leads to the specific killing of tumor cells by immune effector cells while activating immune cells.

Bispecific antibodies can be obtained through a variety of ways, and the preparation methods thereof mainly include chemical coupling method, hybrid-hybridoma method and genetic engineering antibody preparation method. The chemical coupling method comprises preparing a bispecific monoclonal antibody (which is the earliest bispecific monoclonal antibody) by chemically coupling two different monoclonal antibodies together. The hybrid-hybridoma method comprises producing a bispecific monoclonal antibody by means of double hybridoma fusion method or ternary hybridomas. These cell hybridomas or ternary hybridomas are obtained by the fusion of established hybridomas, or the fusion of established hybridomas with lymphocytes from mice and can only be used to produce murine bispecific antibodies, and thus, the application thereof is greatly limited. With the rapid development of molecular biology technology, there have emerged a variety of construction modes of genetically engineered humanized or fully human bispecific antibodies, mainly including four classes of bispecific miniantibodies, double-chain diabodies, single-chain bivalent antibodies, and multivalent bispecific antibodies. At present, several genetically engineered bispecific antibody drugs have entered clinical trials in the world and have shown good application prospects.

The CD3 molecule on the surface of a T cell consists of 4 subunits: 6, £, y and the molecular masses thereof are 18.9 kDa, 23.1 kDa, 20.5 kDa and 18.7 kDa, respectively, and the lengths thereof are 171, 207, 182, and 164 amino acid residues, respectively. They form 6 peptide chains together, which are often tightly bound to a T cell receptor (TCR) to form a TCR-CD3 complex containing 8 peptide chains, and the schematic diagram of the structure thereof is shown in FIG. 1. The complex has the functions of T cell activation, signal transduction and stabilization of the TCR structure. The cytoplasmic segment of CD3 contains immunoreceptor tyrosine-based activation motif (ITAM). TCR recognizes and binds to antigen peptides presented by MHC (major histo-compatibility complex) molecules, rendering the tyrosine residues in the conserved sequence of ITAM of CD3 being phosphorylated by tyrosine protein kinase p561ck in T cells, and then other tyrosine protein kinases (such as ZAP-70) containing SH2 (Scr homology 2) domain being recruited. The phosphorylation of ITAM together with its binding to ZAP-70 is one of the important biochemical reactions in the early stages of the signal transduction process of T cell activation. Therefore, the function of CD3 molecule is to transduce the activation signal generated by recognition of a TCR antigen.

CD19, also known as B4 or Leu-12, belongs to the immunoglobulin (Ig) superfamily, which has a molecular weight of 95 kDa, located on the short arm of chromosome 16, contains 15 exons, and encodes a type I transmembrane glycoprotein of 556 amino acids. When immunoglobulin genes are recombined, CD19 is first expressed in late progenitor B cells and early pre-B cells. CD19 is highly expressed throughout the development and maturation of B cells, and until the B cells differentiate into plasma cells, the expression level is downregulated and its expression in mature B cells is three times that of immature cells.

CD19 establishes B cell signal threshold by simultaneously regulating B cell receptor (BCR) dependent and independent signals, and plays an important regulatory role in the development, proliferation, and differentiation of B cells. As a main component of the surface multi-molecular complex of mature B cells, CD19 forms a complex together with receptors CD21 (CD2), CD81 (TAPA-1) and CD225, and reduces the threshold of antigen concentration required for triggering B cell division and differentiation by regulating endogenous and receptor-induced signals. As a chaperone protein, CD81 provides a molecular docking site for signal transduction pathways and regulates the expression of CD19. CD19 activates protein tyrosine kinase (PTK) by recruiting and amplifying the activation of Src family protein tyrosine kinases and activates BCR signals. Meanwhile, when BCR signals are activated, CD19 can also enhance BCR signals and promote proliferation of B cells by activating PI3K and downstream Akt kinase.

CD19 is expressed both in normal and malignant B lymphocytes and is regarded as one of the most reliable surface markers covering a long period of time during the development of B cells. In normal lymphoid tissues, CD19 is expressed in pre-B cells, B cells and follicular dendritic cells, mantle cells, and dendritic cells in the inter-follicular T cell area. In addition, CD19 can be detected in plasma cells isolated from human tissues through flow cytometry. CD19 is expressed in B lymphocytomas, including B lymphocytic lymphoma, small lymphocytic lymphoma, mantle cell lymphoma, follicular lymphoma, Burkitt lymphoma, and marginal zone lymphoma. Therefore, CD19 has become a specific molecular target for the treatment of B-cell malignant tumors. In recent years, immunotherapy strategies targeting CD19 have been extensively developed in preclinical and clinical studies, including monoclonal antibodies, bispecific antibodies, and chimeric antigen receptor modified T cells (CAR-T), and clinical effects significantly better than conventional small molecule chemotherapy have been achieved, thereby promoting the progress of immunotherapy.

Adoptive immunotherapy for tumors is to inject autologous or allogeneic immunocompetent cells expanded in vitro into a patient to directly kill tumor cells, regulate and enhance the body's immune function, mainly including immunotherapy with LAK cells, TIL cells, activated T lymphocytes and CIK cells. However, immunotherapy can only remove a small number of scattered tumor cells, and has limited efficacy for advanced solid tumors. Therefore, immunotherapy is often used as an adjuvant therapy in combination with conventional methods such as surgery, chemotherapy, radiotherapy and the like. After many tumor cells are firstly cleaned up by conventional methods, then immunotherapy is used to remove remaining tumor cells, and thus the effect of comprehensive tumor treatment can be improved. As a new method in the comprehensive treatment of tumors, adoptive immunotherapy has been widely combined with conventional surgical treatment, radiotherapy, chemotherapy and other cell and molecular therapies, and has broad application prospects in the treatment of various tumors. However, in combination with bispecific antibodies, the ideal adoptive immunotherapy for tumors should be: the bispecific antibody has one end bound to a surface antigen (such as CD3) of immune cells, which is introduced into the body together with the immune cells, while the other end of the bispecific antibody can be well bound to a surface antigen of tumor cells; and in this way, the bispecific antibody can build a bridge between tumor cells and immune cells in the body, so that the immune cells are concentrated around the tumor cells, thereby killing the tumor cells. The metastasis and spread of tumor cells can be effectively solved by this method, which overcomes the disadvantages such as “incomplete, easy to metastasize, and severe side effects” after the three traditional treatments of surgery, radiotherapy and chemotherapy. Therefore, it is of great significance to develop a highly efficient bispecific antibody that binds tumor cells and immune cells.

### SUMMARY OF THE INVENTION

In order to solve the technical problems in the prior art, the purpose of the present invention is to provide a bispecific antibody that binds CD19 and CD3, has a specific targeting effect, and can efficiently stimulate a directed immune response, and a preparation method therefor and use thereof.

In order to achieve the above purpose, the technical solution of the present invention is as follows: by designing and screening of the molecular structure of a bispecific antibody that binds CD19 and CD3, the present invention creatively finds that as compared with corresponding monoclonal antibody and bispecific antibodies with other structures, a bispecific antibody with the following symmetrical structure can better retain the specific binding ability of the original antibody, and meanwhile, has the biological functions of two monoclonal antibodies, and has obvious advantages in terms of production process and medicinal properties: a bispecific antibody comprises (a) a monoclonal antibody unit which consists of two complete light chain-heavy chain pairs, and (b) a single-chain antibody unit comprising two identical single-chain antibodies containing a heavy chain variable region and a light chain variable region, wherein the single-chain antibody unit has the capacity of specifically binding to the surface antigen CD3 of immune cells, the monoclonal antibody unit has the capacity of specifically binding to the surface antigen CD19 of tumor cells, and the single-chain antibody unit is linked to the N-end or C-end of the monoclonal antibody unit through a linker peptide. The present invention has developed a bispecific antibody with the above-mentioned antibody molecular structure that binds CD19 and CD3. This bispecific antibody has a specific targeting effect and can efficiently stimulate a directed immune response and kill tumor cells.

Specifically, firstly, the present invention provides a bispecific antibody, the bispecific antibody comprises (a) a monoclonal antibody unit and (b) a single-chain antibody unit; the monoclonal antibody unit consists of two complete light chain-heavy chain pairs, and can specifically bind to CD19; the single-chain antibody unit comprises two single-chain antibodies (ScFv), and the single-chain antibody comprises a heavy chain variable region and a light chain variable region, and can specifically bind to CD3. The bispecific antibody has a symmetric structure formed by linkage in any one of the following modes:

(1) N-ends of the two single-chain antibodies are respectively linked to C-ends of two heavy chains of the monoclonal antibody through a linker peptide; and

(2) C-ends of the two single-chain antibodies are respectively linked to N-ends of two heavy chains of the monoclonal antibody through a linker peptide.

Preferably, the amino acid sequence of the linker peptide is (GGGGX)n, wherein X is Gly or Ser, and n is a natural number selected from 1 to 4 (that is, 1, 2, 3 or 4). When the linker peptide with the above sequence is used, the bispecific antibody can better perform the antigen-binding function.

As a preferred embodiment of the present invention, the amino acid sequence of the linker peptide is represented by SEQ ID NO. 13.

Preferably, the light chain sequence of the single-chain antibody is represented by SEQ ID NO. 5 or represented by SEQ ID NO. 9.

The heavy chain sequence of the single-chain antibody is represented by SEQ ID NO. 6 or represented by SEQ ID NO. 10.

Both the light chain and the heavy chain of the single-chain antibody can specifically bind to the surface antigen CD3 of immune cells.

In the present invention, the single-chain antibodies are expressed as fusion peptides. Through the specific design of antibody structure and sequence, it is found that when the single-chain antibody and the monoclonal antibody are linked in different ways, the stability of the antibody structure and the binding to two antigens can be better improved by adopting specific fusion peptide sequences of the single-chain antibody, respectively.

For the single-chain antibody unit of the bispecific antibody, preferably, the light chain and the heavy chain of the single-chain antibody constitute a fusion peptide, and the sequence of the fusion peptide is any one of the follows:

(1) when N-ends of the two single-chain antibodies are respectively linked to C-ends of the two heavy chains of the monoclonal antibody through a linker peptide, the sequence of the fusion peptide is represented by SEQ ID NO. 17; and

(2) when C-ends of the two single-chain antibodies are respectively linked to N-ends of two heavy chains of the monoclonal antibody through a linker peptide, the sequence of the fusion peptide is represented by SEQ ID NO. 16.

For the monoclonal antibody unit of the bispecific antibody, preferably, the sequence of the light chain variable region of the monoclonal antibody is represented by SEQ ID NO. 18, or is the amino acid sequence of a polypeptide with the same function which is obtained by subjecting the amino acid sequence represented by SEQ ID NO. 18 to substitution, deletion or insertion of one or more amino acids.

The sequence of the heavy chain variable region of the monoclonal antibody is represented by SEQ ID NO. 19, or is the amino acid sequence of a polypeptide with the same function which is obtained by subjecting the amino acid sequence represented by SEQ ID NO. 19 to substitution, deletion or insertion of one or more amino acids. In the present invention, the bispecific antibody may be a murine antibody, a humanized antibody, a chimeric antibody or a recombinant antibody.

As an embodiment of the present invention, the light chain and the heavy chain of the monoclonal antibody are connected by a disulfide bond. The Fc fragment of the monoclonal antibody is a Fc fragment of a human or humanized antibody.

Preferably, the human or humanized antibody comprises one of IgG1 antibody, IgG2 antibody, IgG3 antibody, and IgG4 antibody.

As a preferred embodiment of the present invention, the Fc fragment of the monoclonal antibody is a Fc fragment of a human or humanized IgG4 antibody.

As a preferred embodiment of the present invention, a full-length sequence of the light chain of the monoclonal antibody is represented by SEQ ID NO. 3; and a full-length sequence of the heavy chain of the monoclonal antibody is represented by SEQ ID NO. 1 or SEQ ID NO. 20.

In the present invention, the above-mentioned “amino acid sequence of a protein with the same function which is obtained by substitution, deletion or insertion of one or more amino acids” refers to a sequence which is different from the shown sequence at one or more amino acid residues but the resulting molecule can retain the biological activity, and it can be a “conservatively modified variant” or obtained by modification through “conservative amino acid substitution”. “Conservatively modified variant” or “conservative amino acid substitution” refers to an amino acid substitution known to a person skilled in the art which generally does not change the biological activity of the obtained molecule. It is acknowledged by a person skilled in the art that the substitution of a single amino acid in the nonessential region of a polypeptide basically does not change the biological activity. Exemplary substitutions are preferably carried out in accordance with the substitutions shown below:

TABLE 1
Exemplary conservative amino acid substitution table

| Original residues | Conservative substitution |
|---|---|
| Ala (A) | Gly, Ser |
| Arg (R) | Lys, His |
| Asn (N) | Gln, His |
| Asp (D) | Glu, Asn |
| Cys (C) | Ser, Ala |
| Gln (Q) | Asn |
| Glu (E) | Asp, Gln |
| Gly (G) | Ala |
| His (H) | Asn, Gln |
| Ile (I) | Leu, Val |
| Lys (K) | Arg, His |
| Met (M) | Leu, Ile, Tyr |
| Phe (F) | Tyr, Met, Leu |
| Pro (P) | Ala |
| Ser (S) | Thr |
| Thr (T) | Ser |
| Trp (W) | Tyr, Phe |
| Tyr (Y) | Trp, Phe |
| Val (V) | Ile, Leu |

As an example of the above-mentioned bispecific antibody, the present invention provides a bispecific antibody against human CD3 and CD19. Among the structures having heavy chain variable region and light chain variable region of the above-mentioned single-chain antibody and heavy chain and light chain of the monoclonal antibody and sequences, two bispecific antibodies binding to CD3 and CD19 that retain the biological function of the corresponding monoclonal antibody to the greatest extent and have obvious advantages in terms of production process and medicinal properties are obtained by screening in the present invention. The structures and sequences of the two bispecific antibodies are as follows:

(1) The sequence of the light chain and heavy chain fusion peptide of the single-chain antibody is represented by SEQ ID NO. 16, the light chain sequence of the monoclonal antibody is represented by SEQ ID NO. 3, and the heavy chain sequence of the monoclonal antibody is represented by SEQ ID NO. 1. The antibody structure is a symmetric structure in which the C-ends of two single-chain antibody fusion peptides are respectively linked to the N-ends of the two heavy chains of the monoclonal antibody through a linker peptide represented by SEQ ID NO. 13 (as shown in FIG. 2 A).

(2) The sequence of the light chain and heavy chain fusion peptide of the single-chain antibody is represented by SEQ ID NO. 17, the light chain sequence of the monoclonal antibody is represented by SEQ ID NO. 3, and the heavy chain sequence of the monoclonal antibody is represented by SEQ ID NO. 20. The antibody structure is a symmetric structure in which the N-ends of two single-chain antibody fusion peptides are respectively linked to the C-ends of the two heavy chains of the monoclonal antibody through the linker peptide represented by SEQ ID NO. 13 (as shown in FIG. 2 B).

Based on the above-mentioned amino acid sequences of the bispecific antibody, the present invention also provides a gene encoding the bispecific antibody.

According to the codon coding rules and the degeneracy and preference of the codon, a person skilled in the art can design the coding gene according to the above-mentioned amino acid sequences of the bispecific antibody.

As a preferred embodiment of the present invention, a gene sequence coding the full-length light chain of the monoclonal antibody is represented by SEQ ID NO. 4.

As a preferred embodiment of the present invention, a gene sequence coding the full-length heavy chain of the monoclonal antibody is represented by SEQ ID NO. 2 or represented by SEQ ID NO. 21.

As a preferred embodiment of the present invention, when the C-ends of the two single-chain antibodies are respectively linked to the N-ends of the two heavy chains of the monoclonal antibody through a linker peptide, a gene sequence coding the single-chain antibody is represented by SEQ ID NO. 14; and when the N-ends of the two single-chain antibodies are respectively linked to the C-ends of the two heavy chains of the monoclonal antibody through a linker peptide, a gene sequence coding the single-chain antibody is represented by SEQ ID NO. 15.

The above-mentioned gene sequences can be combined to express the bispecific antibody or can be respectively combined with other coding gene sequences of the remaining units of the bispecific antibody to express the bispecific antibody.

Further, the present invention also provides a biological material comprising the above-mentioned gene.

In the present invention, the biological material comprises a recombinant DNA, an expression cassette, a vector, a host cell, an engineered bacterium or cell line.

The present invention also provides a preparation method of the bispecific antibody, comprising: constructing an expression vector containing coding genes of the single-chain antibody and the monoclonal antibody; introducing the expression vector into a host cell to obtain a host cell stably expressing the bispecific antibody; culturing the host cell, and obtaining the bispecific antibody by separation and purification.

When preparing the bispecific antibody, a person skilled in the art can select the host cell, expression vector, method for introducing the expression vector into the host cell and separation and purification method of the antibody that are conventional in the art as needed.

As an embodiment of the present invention, the host cell is CHO-K1 cell.

As an embodiment of the present invention, the expression vector is pG4HK.

The construction of the expression vector can use conventional methods in the art. As a preferred embodiment of the present invention, the construction method of the expression vector comprises: linking the light chain coding gene of the anti-CD19 monoclonal antibody to an expression vector pG4HK by double enzyme digestion with SalI and BsiWI to obtain an expression vector of anti-CD19 light chain named as pG4HK19VL; and linking the fusion fragment of the anti-CD3 single-chain antibody coding gene and the heavy chain gene of the anti-CD19 monoclonal antibody to a vector pG4HK19VL by double enzyme digestion with Hind III and BstEII to obtain a bispecific antibody expression vector.

The separation and purification can be performed by antibody separation and purification method commonly used in the art.

As an embodiment of the present invention, the separation and purification comprises the following steps:

(1) separating all antibodies with Fc domain from a culture supernatant through a recombinant rProtein A affinity chromatography column;

(2) separating a bispecific antibody from by-products by anion exchange Q-Sepharose column chromatography; and

(3) purifying the bispecific antibody by molecular sieve chromatography.

Based on the above-mentioned bispecific antibody, the present invention also provides a pharmaceutical composition comprising the bispecific antibody of the present invention.

Preferably, the pharmaceutical composition also comprises other pharmaceutically acceptable active ingredients or adjuvants.

Further, the present invention provides any one of the following uses of the bispecific antibody or the coding gene of the bispecific antibody or the biological material comprising the coding gene:

(1) use in the preparation of a drug for prevention or treatment of CD19-expressing B cell-related diseases;

(2) use in the preparation of a drug for prevention or treatment of a disease with CD19 as a target;

(3) use in the preparation of a drug for killing CD19-expressing cells; and

(4) use in the preparation of a detection reagent for CD19 and/or CD3.

In the present invention, the CD19-expressing B cell-related diseases include but are not limited to B cell-related tumors and autoimmune diseases caused by B cells.

The B cell-related tumors are not limited to B-lymphocytoma and B-lineage leukemia.

The beneficial effects of the present invention are as follows:

By genetic engineering and antibody engineering methods, the present invention constructs a bispecific antibody that comprises a single-chain antibody and a complete monoclonal antibody structure and binds to CD19 and CD3. The bispecific antibody fusion protein retains a complete monoclonal antibody structure, and has a highly stable symmetrical structure, better retains the biological functions of an anti-CD3 single-chain antibody and an anti-CD19 monoclonal antibody, realizes a bispecific antibody molecule simultaneously having excellent biological functions of anti-CD19 and anti-CD3 monoclonal antibodies, which can build a bridge between tumor cells and immune effector cells, effectively activate immune effector cells and directed immune responses, significantly enhance the efficacy of immune cells to kill tumor cells, and minimize the ADCC effect, with high safety. In addition, because the bispecific antibody provided by the present invention has a feature of completely symmetrical structure, when expressed in the host, no protein isomers of other structures will be produced, thus the difficulty of extraction and purification process is greatly reduced. The bispecific antibody has the advantages of simple preparation and high yield and has broad application prospects in tumor immunotherapy.

### BRIEF DESCRIPTION OF THE DRAWINGS

FIG. 1 is a schematic diagram of the structure of the cell surface antigen CD3 molecule in the background of the present invention.

FIG. 2 is a schematic diagram of the molecular structures of two bispecific antibodies YK001 and YK002 obtained through screening in Example 1 of the present invention, wherein A represents the bispecific antibody YK001; and B represents the bispecific antibody YK002.

FIG. 3 is the SDS-PAGE electrophoresis diagram of the bispecific antibodies YK001 and YK002 in Example 2 of the present invention, wherein A and C represent reduced SDS-PAGE electrophoresis detection; B and D represent non-reduced SDS-PAGE electrophoresis detection; A and B represent SDS-PAGE electrophoresis results of YK001 bispecific antibody; C and D represent SDS-PAGE electrophoresis results of YK002 bispecific antibody; M represents protein molecular weight marker, and lane 1 represents the target protein.

FIG. 4 shows HPLC-SEC purity peak graphs of bispecific antibodies YK001 and YK002 in Example 2 of the present invention, wherein A represents the bispecific antibody YK001; and B represents the bispecific antibody YK002.

FIG. 5 shows the binding efficiency of bispecific antibodies YK001 and YK002 with Raji cells determined based on flow cytometry in Example 3 of the present invention, wherein A represents the negative control NC; B represents the bispecific antibody YK001; C represents the positive control antibody (PC) Anti-CD19; D represents the negative control NC; E represents the bispecific antibody YK002; and F represents the positive control antibody (PC) Anti-CD19.

FIG. 6 shows the binding efficiency of bispecific antibodies YK001 and YK002 with T cells determined based on flow cytometry in Example 3 of the present invention, wherein A represents the negative control NC; B represents the bispecific antibody YK001; C represents the bispecific antibody YK002, and D represents the positive control (PC) Anti-CD3.

FIG. 7 is a diagram showing the results in Example 4 of the present invention that the bispecific antibodies YK001 and YK002 effectively mediated PBMC cells to kill Raji tumor cells, wherein (▾) represents the bispecific antibody YK001, (∇) represents the bispecific antibody YK002, (▪) represents Anti-CD19 monoclonal antibody, (⋄) represents irrelevant control 0527×CD3 bispecific antibody (Her2×CD3 bispecific antibody), and (●) represents Anti-CD3 monoclonal antibody.

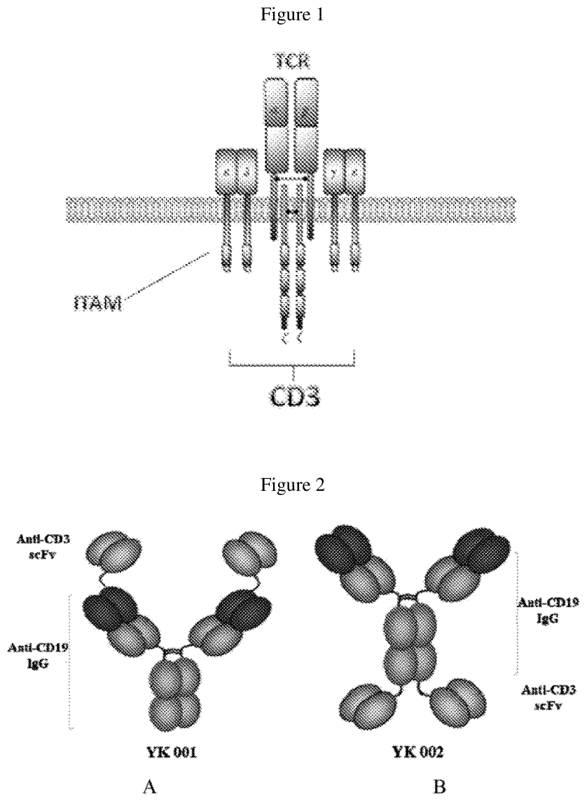

### SPECIFIC MODES FOR CARRYING OUT THE EMBODIMENTS

The preferred embodiments of the present invention will be described in detail below in conjunction with Examples. It should be understood that the following Examples are given for illustrative purposes only and are not intended to limit the scope of the present invention. A person skilled in the art can make various modifications and alternatives to the present invention without departing from the aim and spirit of the present invention.

The experimental methods used in the following examples are conventional methods unless otherwise specified.

The materials and reagents used in the following Examples can be obtained from commercial sources unless otherwise specified.

### Example 1: Design of the Structure and Sequence of CD19×CD3 Bispecific Antibody

In the present Example, the tumor cell surface antigen CD19 and the immune cell surface antigen CD3 were used as targets to design a bispecific antibody.

Combined with protein structure design software and a lot of artificial experimental screening, a variety of CD19 and CD3 binding bispecific antibody structures were screened in the present invention for bispecific antibody structures with symmetrical structures comprising a single-chain antibody unit and a monoclonal antibody unit, wherein the anti-CD19 monoclonal antibody unit is an IgG antibody, and comprises two complete light chain-heavy chain pairs (i.e., containing complete Fab and Fc domains, and the heavy chain and the light chain are connected by a disulfide bond), the anti-CD3 single-chain antibody unit comprises two single-chain antibodies (ScFv), each single-chain antibody contains a heavy chain variable region domain and a light chain variable region domain, and the heavy chain variable region and the light chain variable region are constructed as a fusion peptide through a linker peptide. The single-chain antibody and the monoclonal antibody are linked by a linker peptide. For the linkage modes between the single-chain antibody and the monoclonal antibody, two different linkage methods were designed to obtain two bispecific antibodies with different symmetric structures:

(1) The C-ends of the anti-CD3 single-chain antibody were linked to the N-ends of the heavy chain of the anti-CD19 monoclonal antibody through a linker peptide of GGGGSGGGGSGGGGS (represented by SEQ ID NO. 13) to obtain a bispecific antibody YK001 (with the structure schematic diagram as shown in FIG. 2 A); and

(2) The N-ends of the anti-CD3 single-chain antibody were linked to the C-ends of the heavy chain of the anti-CD19 monoclonal antibody through a linker peptide of GGGGSGGGGSGGGGS (represented by SEQ ID NO. 13) to obtain a bispecific antibody YK002 (with the structure schematic diagram as shown in FIG. 2 B).

The amino acid sequence of each domain of the above-mentioned bispecific antibody is as follows:

The amino acid sequence of the heavy chain variable region of the anti-CD19 monoclonal antibody of YK001 is represented by SEQ ID NO. 19, and the amino acid sequence of the full-length heavy chain is represented by SEQ ID NO. 1.

The amino acid sequence of the heavy chain variable region of the anti-CD19 monoclonal antibody of YK002 is represented by SEQ ID NO. 19, and the amino acid sequence of the full-length heavy chain is represented by SEQ ID NO. 20.

The amino acid sequence of the light chain variable region of the anti-CD19 monoclonal antibody is represented by SEQ ID NO. 18, and the amino acid sequence of the full-length light chain is represented by SEQ ID NO. 3 (same for YK001 and YK002).

The amino acid sequence of the anti-CD3 single-chain antibody in YK001 is represented by SEQ ID NO. 16.

The amino acid sequence of the anti-CD3 single-chain antibody in YK002 is represented by SEQ ID NO. 17.

### Example 2: Preparation of a CD19×CD3 Bispecific Antibody

1. Design and synthesis of coding genes of the bispecific antibody

According to the amino acid sequences of the two bispecific antibodies YK001 and YK002 obtained by the design and screening in Example 1, and the codon preference of the host cell, the coding genes of the bispecific antibodies were designed, with the specific sequences as follows:

the nucleotide sequence coding the heavy chain of the anti-CD19 monoclonal antibody of YK001 was represented by SEQ ID NO. 2;

the nucleotide sequence coding the heavy chain of the anti-CD19 monoclonal antibody of YK002 was represented by SEQ ID NO. 21;

the nucleotide sequence coding the light chain of the anti-CD19 monoclonal antibody was represented by SEQ ID NO. 4 (same for YK001 and YK002);

the nucleotide sequence coding the anti-CD3 single-chain antibody in YK001 was represented by SEQ ID NO. 14; and

the nucleotide sequence coding the anti-CD3 single-chain antibody in YK002 was represented by SEQ ID NO. 15.

In order to facilitate the construction of expression vectors, the gene fragment coding the light chain of the anti-CD19 monoclonal antibody (same for YK001 and YK002) and the fusion fragment of the coding gene of the anti-CD3 single-chain antibody and the coding gene of the heavy chain of the anti-CD19 monoclonal antibody (YK001, i.e., the C-end of the anti-CD3 single-chain antibody is linked to the N-end of the heavy chain of the anti-CD19 monoclonal antibody) and the fusion fragment of the coding gene of the heavy chain of the anti-CD19 monoclonal antibody and the coding gene of the anti-CD3 single-chain antibody (YK002, i.e., the N-end of the anti-CD3 single-chain antibody is connected to the C-end of the heavy chain of the anti-CD19 monoclonal antibody) were synthesized.

2. Construction of Bispecific Antibody Expression Vectors

(1) The coding gene of the light chain of the anti-CD19 monoclonal antibody was linked to the expression vector pG4HK by double enzyme digestion with SalI and BsiWI to obtain the expression vector of the light chain of the anti-CD19 monoclonal antibody named as pG4HK19VL.

(2) The fusion fragment of the coding gene of the anti-CD3 single-chain antibody and the coding gene of the heavy chain of the anti-CD19 monoclonal antibody was linked to the vector pG4HK19VL by double enzyme digestion with Hind III and BstE II to obtain the YK001 bispecific antibody expression vector name as pG4HK-YK001.

(3) The fusion fragment of the coding gene of the heavy chain of the anti-CD19 monoclonal antibody and the coding gene of the anti-CD3 single-chain antibody coding gene was linked to the vector pG4HK19VL by double enzyme digestion with Hind III and BstE II to obtain the YK002 bispecific antibody expression vector named as pG4HK-YK002.

3. Expression of Bispecific Antibodies

(1) Plasmid was subject to large-scale extraction with an endotoxin-free large-scale extraction kit (Qiagen, 4991083), and the specific operation was carried out according to the instructions of the kit.

(2) Preparation of Cells for Transfection

(i) CHO-K1 cells were resuscitated, 6×10⁶ cells were inoculated into 12 ml CD-CHO medium (containing 6 mM GlutaMAX) at a density of 0.5×10⁶/ml, and the resultant was subjected to shake cultivation in 5% CO₂, at 37° C. and 135 rpm.

(ii) On the day before transfection, the cell density was adjusted to 0.5×10⁶/ml, and the resultant was subjected to shake cultivation in 5% CO₂, at 37° C. and 135 rpm.

(3) Electroporation transfection

(i) The cell concentration was measured by cell counting to ensure a cell viability of 95% or more.

(ii) 1×10⁷ cells were taken, centrifuged at 1,000 rpm for 5 min, the supernatant was discarded, the cells were suspended with fresh CD-CHO medium, the resultant was centrifuged at 1,000 rpm for 5 min, and the supernatant was discarded. Washing was repeated once again.

(iii) Cells were suspended with 0.7 ml CD-CHO medium, 40 μg of expression vector was added to be mix welled and the resultant was transferred to a 0.4 cm electroporation cuvette for electroporation.

(iv) The cells were quickly transferred to CD-CHO medium (without GlutaMAX) after electroporation, and plated in a 96-well plate, and cultured in 5% CO₂ at 37° C.

(v) 24 hours after transfection, MSX was added to each well to a final concentration of 50 μM, and the resultant was subjected to cultivation in 5% CO₂ at 37° C.

(vi) Monoclonal cell strains that highly express bispecific antibodies were picked out to perform fed-batch fermentation and the supernatant was collected after 14 days of culturing.

4. Purification of Bispecific Antibodies

(1) Pretreatment of Feed

The supernatant of the fermentation culture was centrifuged at 2,000 rpm for 10 min, and then filtered with a 0.22 μM filter membrane.

(2) Affinity Chromatography

A Mabselect SuRe affinity chromatography column (purchased from GE, Catalog No. 18-5438-02) was used to capture the antibodies in the pretreated fermentation broth, an equilibration buffer (10 mM PB, 0.1 M NaCl, pH 7.0) was used to fully equilibrate the chromatography column, and the pretreated fermentation broth was allowed to pass through the affinity chromatography column, and elution was performed with an elution buffer (0.1 M citric acid, pH 3.0).

(3) Cation Exchange Chromatography

The sample prepared by affinity chromatography was further subjected to purification by SP cation exchange chromatography. The cation exchange column was purchased from GE (17-1014-01, 17-1014-03). After equilibration of the chromatography column with an equilibration buffer (50 mM PBS, pH 5.5), the sample was allowed to pass through the SP column for binding, and then linear elution was performed with 20 column volumes of an elution buffer (50 mM PBS, 1.0 M NaCl, pH 5.5).

(4) Anion Exchange Chromatography

After purification by SP cation exchange chromatography, the resultant was further allowed to pass through an ion exchange Q-Sepharose column (purchased from GE, Catalog Nos: 17-1153-01, 17-1154-01), and the buffer used was 50 mM PBS at pH 5.5.

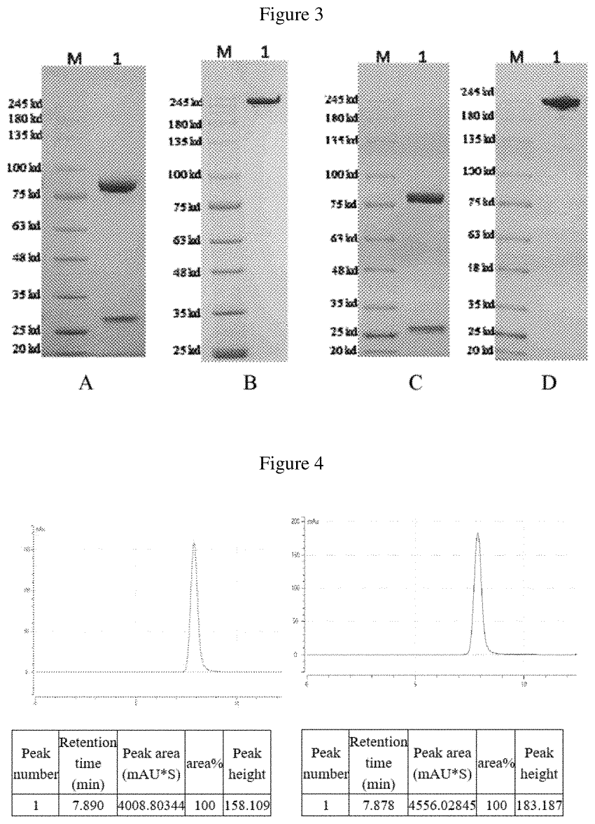

The purified bispecific antibodies YK001 and YK002 were tested by SDS-PAGE and HPLC-SEC. The result of SDS-PAGE is shown in FIG. 3, the test result of reduced SDS-PAGE electrophoresis of YK001 is shown in A of FIG. 3, and the test result of non-reduced SDS-PAGE electrophoresis of YK001 is shown in B of FIG. 3. The test result of reduced SDS-PAGE electrophoresis of YK002 is shown in C of FIG. 3, and the test result of non-reduced SDS-PAGE electrophoresis of YK002 is shown in D of FIG. 3. The test result of HPLC-SEC is shown in FIG. 4, wherein the SEC test result of YK001 is shown in A of FIG. 4, and the SEC test result of YK002 is shown in B of FIG. 4. The test results show that the bispecific antibodies YK001 and YK002 are successfully prepared after expression and purification, and the purity of the purified bispecific antibodies is 95% or more.

### Example 3: Determination of the Binding Activity of Bispecific Antibodies to Tumor Cells and Immune Cells

Raji cells (purchased from ATCC, CCL-86) were used as CD19-positive cells, T cells were used as CD3-positive cells, and the binding activity of the bispecific antibody of the present invention to target antigens of CD19-expressing tumor cells and CD3-expressing immune cells was detected by flow cytometry.

1. Detection of the binding activity of bispecific antibodies to Raji cells by flow cytometry

(1) Collecting Raji cells: cells were collected at 1×10⁶ cells/tube.

(2) Rinsing the cells: the cells were rinsed once with 1 ml staining buffer (PBS containing 0.5% w/v BSA+2 mM EDTA), the resultant was centrifuged at 350×g at 4° C. for 5 min, and then cells were resuspended with 200 μl staining buffer.

(3) Bs-antibody binding: bispecific antibodies YK001 and YK002 were added to a concentration of 5 μg/ml, respectively, and the resultant was subjected to incubation on ice for 45 min.

(4) Rinsing the cells: 1 ml staining buffer was added to the cell suspension to mix well, and centrifuged at 350×g at 4° C. for 5 min, the supernatant was removed, and the resultant was rinsed once again. After centrifugation, cells were resuspended with 100 μl staining buffer.

(5) 5 μl of Biolegend antibody (PE anti-human IgG Fc Antibody, Biolegend, 409304) was added to a sample tube, isotype control (PE Mouse IgG2a, κ Isotype Ctrl (FC) Antibody, Biolegend, 400213) was added to an isotype control tube, and the resultants were subjected to incubation on ice in dark for 15 min.

(6) Rinsing the cells: 1 ml staining buffer was added to the cell suspension to mix well, the resultant was centrifuged at 350×g at 4° C. for 5 min, the supernatant was removed, and the resultant was rinsed once again.

(7) Detection with a flow cytometer: After resuspending the cells with 200 μl PBS, the resultant was subjected to detection with a flow cytometer.

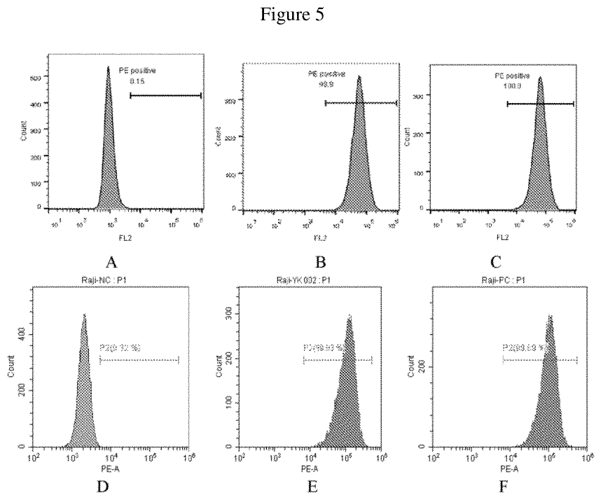

The results of flow cytometry were shown in FIG. 5, wherein the detection results of binding of YK001 to Raji cells are shown in A, B and C of FIG. 5, and the detection results of binding of YK002 to Raji cells are shown in D, E and F of FIG. 5. The results show that both bispecific antibodies YK001 and YK002 can specifically bind to Raji cells, that is, the bispecific antibody fusion protein retains the binding function of the monoclonal antibody Anti-CD19.

2. Detection of the binding activity of bispecific antibodies to T cells by means of flow cytometry

(1) Collecting T cells: cells were collected at 1×10⁶ cells/tube.

(2) Rinsing the cells: the cells were rinsed once with 1 ml staining buffer (PBS containing 0.5% w/v BSA+2 mM EDTA), the resultant was centrifuged at 350×g at 4° C. for 5 min, and then the cells were resuspended with 200 μl staining buffer.

(3) Bs-antibody binding: bispecific antibodies YK001 and YK002 were added to a concentration of 5 μg/ml, respectively, and the resultant was subjected to incubation on ice for 45 min.

(4) Rinsing the cells: 1 ml staining buffer was added to the cell suspension to mix well, the resultant was centrifuged at 350×g at 4° C. for 5 min, the supernatant was removed, and the resultant was rinsed once again. After centrifugation, the cells were resuspended with 100 μl staining buffer.

(5) 5 μl of Biolegend antibody (PE anti-human IgG Fc Antibody, Biolegend, 409304) was added to a sample tube, isotype control (PE Mouse IgG2a, κ Isotype Ctrl (FC) Antibody, Biolegend, 400213) was added to an isotype control tube, and the resultants were subjected to incubation on ice in dark for 15 min.

(6) Rinsing the cells: 1 ml staining buffer was added to the cell suspension to mix well, the resultant was centrifuged at 350×g at 4° C. for 5 min, the supernatant was removed, and the resultant was rinsed once again.

(7) Detection with a flow cytometer: After resuspending the cells with 200 μl PBS, the resultant was subjected to detection with a flow cytometer.

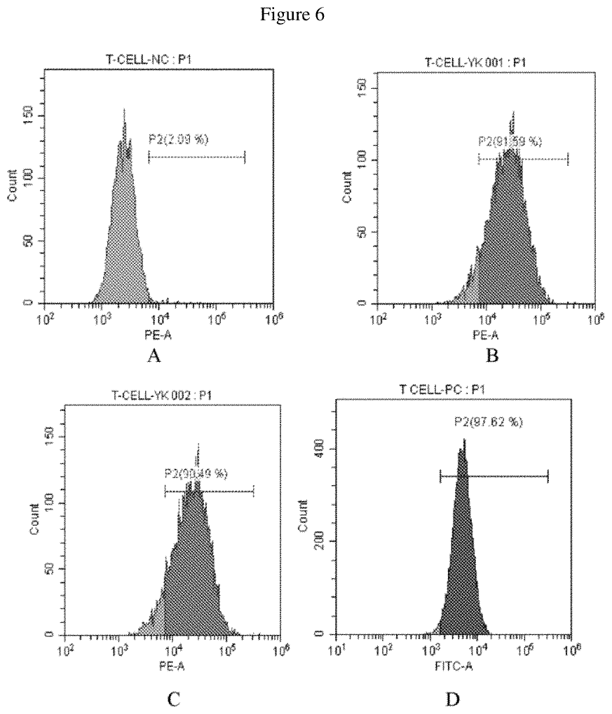

The results of flow cytometry were shown in FIG. 6, wherein the detection results of binding of YK001 to T cells are shown in A and B of FIG. 6, and the detection results of binding of YK002 to T cells are shown in C and D of FIG. 6. The results show that both bispecific antibodies YK001 and YK002 can specifically bind to T cells, that is, the bispecific antibody fusion protein retains the binding function of the single-chain antibody Anti-CD3.

### Example 4: Detection of In-Vitro Cell Killing Efficiency Mediated by Bispecific Antibodies

In the present Example, Raji-Luc cells were used as target cells, PBMCs were used as immune effector cells, and the effect of killing the target cells mediated by bispecific antibodies YK001 and YK002 was detected, with anti-CD3 monoclonal antibody and anti-CD19 monoclonal antibody and 0527×CD3 bispecific antibody as control.

1. Preparation of Target Cells

As target cells, Raji-Luc cells (luciferase-labeled Raji cells) were counted after mixing well by pipetting up and down, centrifuged at 1,000 rpm for 5 min, and washed once with PBS. After centrifugation and washing of the target cells, the density was adjusted to 0.2×10⁶/ml with GT-T551 culture medium, 50 μl of the resultant was added to each well with 10,000 cells in each well.

2. Preparation of PBMCs

PBMCs were used as effector cells. PBMCs frozen in a liquid nitrogen tank were taken out (referring to cell cryopreservation and resuscitation), thawed and added to a 15 ml centrifuge tube containing PBS or GT-T551 culture medium, and centrifuged at 1,000 rpm for 5 min. The cells were washed twice with PBS or GT-T551 culture medium and counted, the activity and density of cells were detected, and the density of living cells was adjusted to 2×10⁶/ml. 50 μl of the resultant was added to each well with 100,000 cells in each well.

3. Dilution of Antibodies

The bispecific antibodies YK001 and YK002 were diluted with GT-T551 culture medium, respectively, and the initial concentration of the antibodies YK001 and YK002 was adjusted to 10 nM. The resultant was diluted sequentially at a ratio of 1:5. 100 μl of the diluted antibody was added to the cells prepared above in a 96-well plate to mix well, the 96-well plate was put back into the incubator, and the killing effect was detected after 18 hours.

4. Detection

Since the Luciferase gene was carried by Raji target cells, the efficiency of killing the target cells was detected by the LUMINEX method.

Steady-GLO (Promega) was used as a substrate. After thawed, the buffer in the kit was added to the substrate powder to mix well, and the resultant was sub-packed with 5 ml or 10 ml for each package to complete reconstruction of the steady-GLO substrate.

After the co-cultured cells were mixed well by pipetting up and down, 100 μl was taken and transferred to an opaque white plate, then 100 μl of the reconstructed steady-GLO substrate was added, the resultant was tapped to mix well, and detected by a plate reader after standing for 5 minutes. The detection instrument was synergy HT.

5. Data Processing

The calculation formula for the killing ratio of the target cell is as follows:

Killing ratio of target cells=100×(Only target−test well)/Only target.

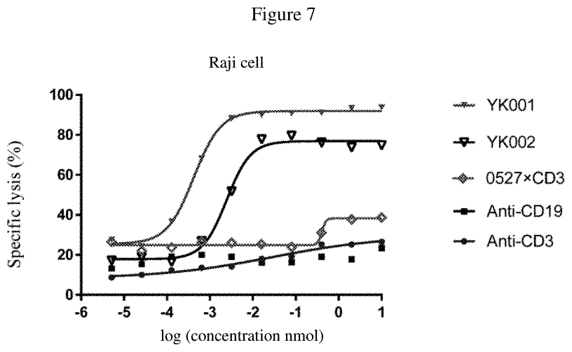

The antibody concentration corresponding to the killing ratio of target cells in all detection wells was converted to log 10, which was used as the abscissa, and the killing ratio was used as the ordinate to make a graph. The results were shown in FIG. 7. The results were analyzed by the software Graphpad Prism 7.0, the IC50 of the bispecific antibody was calculated, and the results were shown in Table 2. The results show that compared with control antibodies (anti-CD3 monoclonal antibody, anti-CD19 monoclonal antibody and 0527×CD3 bispecific antibody), both bispecific antibodies YK001 and YK002 can effectively mediate PBMC to kill the tumor cell line Raji-Luc, as a single molecular, both YK001 and YK002 have the biological functions of Anti-CD19 and Anti-CD3 monoclonal antibodies at the same time, and the efficacy of killing target cells mediated by YK001 is higher than that of YK002.

TABLE 2
IC50 of target cell killing mediated by
bispecific antibodies YK001 and YK002

| | Anti- | Anti- | | | 0527 × |
|---|---|---|---|---|---|
| | CD3 | CD19 | YK001 | YK002 | CD3 |
| IC50 (nM) | 0.02866 | N/A | 0.0004193 | 0.002445 | ~0.4051 |

Although the general description and specific embodiments have been used to describe the present invention in detail above, it is obvious to a person skilled in the art that some modifications or improvements can be made based on the present invention. Therefore, these modifications or improvements made without departing from the spirit of the present invention fall within the scope of protection of the present invention.

### INDUSTRIAL APPLICABILITY

The present invention provides a bispecific antibody, a preparation method thereof and a use thereof. The bispecific antibody of the present invention comprises a monoclonal antibody unit and a single-chain antibody unit, wherein, the monoclonal antibody unit comprises two complete light chain-heavy chain pairs, and can specifically bind to a surface antigen of a tumor cell; the single-chain antibody unit comprises two single-chain antibodies, and the single-chain antibody comprises a heavy chain variable region and a light chain variable region, and can specifically bind to a surface antigen of an immune cell. The bispecific antibody provided in the present invention is of a symmetric structure formed by linkage in any one of the following modes: (1) C-ends of the two single-chain antibodies are respectively linked to N-ends of two heavy chains of a monoclonal antibody through a linker peptide; (2) N-ends of the two single-chain antibodies are respectively linked to C-ends of the two heavy chains of the monoclonal antibody through a linker peptide. The bispecific antibody of the present invention can simultaneously bind to the immune cell and the tumor cell, mediate a directed immune response, and effectively kill the tumor cell, with good economic value and application prospects.

1. A bispecific antibody that binds to CD19 and CD3, wherein the bispecific antibody comprises (a) a monoclonal antibody unit and (b) a single-chain antibody unit; the monoclonal antibody unit consists of two complete light chain-heavy chain pairs, and can specifically bind to CD19; the single-chain antibody unit comprises two single-chain antibodies; the single-chain antibody comprises a heavy chain variable region and a light chain variable region, and can specifically bind to CD3; the bispecific antibody has a symmetric structure formed by linkage in any one of the following modes:
(1) the C-ends of the two single-chain antibodies are respectively linked to the N-ends of two heavy chains of the monoclonal antibody through a linker peptide; and
(2) N-ends of the two single-chain antibodies are respectively linked to C-ends of the two heavy chains of the monoclonal antibody through a linker peptide.

2. The bispecific antibody according to claim 1, wherein the amino acid sequence of the linker peptide is (GGGGX)n, wherein X is Gly or Ser, and n is a natural number selected from 1 to 4;
preferably, the amino acid sequence of the linker peptide is represented by SEQ ID NO. 13.

3. The bispecific antibody according to claim 1, wherein,
the light chain sequence of the single-chain antibody is represented by SEQ ID NO. 5 or represented by SEQ ID NO. 9;
the heavy chain sequence of the single-chain antibody is represented by SEQ ID NO. 6 or represented by SEQ ID NO. 10;
preferably, the light chain and the heavy chain of the single-chain antibody constitute a fusion peptide, and the sequence of the fusion peptide is any one of the follows:
(1) when C-ends of the two single-chain antibodies are respectively linked to N-ends of two heavy chains of the monoclonal antibody through a linker peptide, the sequence of the fusion peptide is represented by SEQ ID NO. 16; and
(2) when N-ends of the two single-chain antibodies are respectively linked to to C-ends of the two heavy chains of the monoclonal antibody through a linker peptide, the sequence of the fusion peptide is represented by SEQ ID NO. 17.

4. The bispecific antibody according to claim 3, wherein the bispecific antibody is a murine antibody, a humanized antibody, a chimeric antibody or a recombinant antibody.

5. The bispecific antibody according to claim 3, wherein the light chain and the heavy chain of the monoclonal antibody are connected by a disulfide bond;
Fc fragment of the monoclonal antibody is a Fc fragment of a human or humanized antibody, and the human or humanized antibody is one of IgG1, IgG2, IgG3 or IgG4;
preferably, the Fc fragment of the monoclonal antibody is a Fc fragment of a human or humanized IgG4 antibody;
more preferably, a full-length sequence of the light chain of the monoclonal antibody is represented by SEQ ID NO. 3; and a full-length sequence of the heavy chain of the monoclonal antibody is represented by SEQ ID NO. 1 or SEQ ID NO. 20.

6. A gene encoding the bispecific antibody claim 1,
preferably, a gene sequence coding a full-length light chain of the monoclonal antibody is represented by SEQ ID NO. 4; and/or,
a gene sequence coding a full-length heavy chain of the monoclonal antibody is represented by SEQ ID NO. 2 or represented by SEQ ID NO. 21; and/or,
when C-ends of the two single-chain antibodies are respectively linked to N-ends of two heavy chains of the monoclonal antibody through a linker peptide, a gene sequence coding the single-chain antibody is represented by SEQ ID NO. 14; and when N-ends of the two single-chain antibodies are respectively linked to C-ends of the two heavy chains of the monoclonal antibody through a linker peptide, a gene sequence coding the single-chain antibody is represented by SEQ ID NO. 15.

7. A biological material comprising the gene of claim 6, wherein the biological material comprises a recombinant DNA, an expression cassette, a vector, a host cell, an engineered bacterium or a cell line.

8. A preparation method of the bispecific antibody according to claim 1, wherein the method comprises: constructing an expression vector containing a coding gene of the single-chain antibody and the monoclonal antibody; introducing the expression vector into a host cell to obtain a host cell stably expressing the bispecific antibody; culturing the host cell, and obtaining the bispecific antibody by separation and purification.

9. A pharmaceutical composition, wherein the pharmaceutical composition comprises the bispecific antibody of claim 1.

10. (canceled)

11. The bispecific antibody according to claim 2, wherein,
the light chain sequence of the single-chain antibody is represented by SEQ ID NO. 5 or represented by SEQ ID NO. 9;
the heavy chain sequence of the single-chain antibody is represented by SEQ ID NO. 6 or represented by SEQ ID NO. 10;
preferably, the light chain and the heavy chain of the single-chain antibody constitute a fusion peptide, and the sequence of the fusion peptide is any one of the follows:
(1) when C-ends of the two single-chain antibodies are respectively linked to N-ends of two heavy chains of the monoclonal antibody through a linker peptide, the sequence of the fusion peptide is represented by SEQ ID NO. 16; and
(2) when N-ends of the two single-chain antibodies are respectively linked to C-ends of the two heavy chains of the monoclonal antibody through a linker peptide, the sequence of the fusion peptide is represented by SEQ ID NO. 17.

12. The bispecific antibody according to claim 4, wherein the light chain and the heavy chain of the monoclonal antibody are connected by a disulfide bond;
Fc fragment of the monoclonal antibody is a Fc fragment of a human or humanized antibody, and the human or humanized antibody is one of IgG1, IgG2, IgG3 or IgG4;
preferably, the Fc fragment of the monoclonal antibody is a Fc fragment of a human or humanized IgG4 antibody;
more preferably, a full-length sequence of the light chain of the monoclonal antibody is represented by SEQ ID NO. 3; and a full-length sequence of the heavy chain of the monoclonal antibody is represented by SEQ ID NO. 1 or SEQ ID NO. 20.

13. The pharmaceutical composition according to claim 9, wherein the pharmaceutical composition is used for prevention or treatment of a CD19-expressing B cell-related disease;
preferably, the CD19-expressing B cell-related disease include B cell-related tumors and autoimmune diseases caused by B cells.

14. The pharmaceutical composition according to claim 9, wherein the pharmaceutical composition is used for prevention or treatment of a disease with CD19 as a target.

15. The pharmaceutical composition according to claim 9, wherein the pharmaceutical composition is used for killing CD19-expressing cells.

16. The pharmaceutical composition according to claim 9, wherein the pharmaceutical composition is used as a detection reagent for CD19 and/or CD3.


SEQUENCE LISTING (21 sequences)

SEQ ID NO: 1 - protein, 451 aa, Artificial Sequence
  Laboratory synthesis
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 2 - DNA, 1353 bp, Artificial Sequence
  Laboratory synthesis
caagttcaacttcagcagtctggcgccgaactcgtgcggcctggatctagcgtgaagatcagctgtaaagccagcggctacgccttcagcagctactggatgaactgggtcaagcagaggcctggacagggcctcgaatggatcggacaaatttggcctggcgacggcgacaccaactacaacggcaagttcaagggcaaagccacactgaccgccgacgagtctagcagcacagcctacatgcagctgagcagcctggcctctgaagatagcgccgtgtacttctgcgccagacgggaaacaaccaccgtgggcagatattactacgccatggactactggggccagggcaccacagtgacagttagctctgcgtcgaccaagggccccagcgtgttccccctggccccttgcagcagaagcaccagcgagagcacagccgccctgggctgcctggtgaaggactacttccccgagcccgtgaccgtgtcctggaacagcggcgctctgaccagcggcgtgcataccttccccgccgtgctccagagcagcggactgtactccctgagcagcgtggtgaccgtgccttccagcagcctgggcaccaagacctacacttgcaacgtggaccacaagcccagcaacaccaaggtggacaagagagtggagagcaagtacggccccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaa

SEQ ID NO: 3 - protein, 218 aa, Artificial Sequence
  Laboratory synthesis
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - DNA, 657 bp, Artificial Sequence
  Laboratory synthesis
gatatccagctgacacagagccctgccagcctggccgtttctctgggacagagagccaccatcagctgcaaggccagccagagcgttgactacgacggcgacagctacctgaactggtatcagcagatccccggccagcctcctaagctgctgatctacgatgccagcaacctggtgtctggcatccctcctagattttccggcagcggctctggcaccgacttcaccctgaatatccatcctgtggaaaaggtggacgccgccacctaccactgtcagcagtctacagaggacccctggacatttggcggaggcaccaagctggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 5 - protein, 106 aa, Artificial Sequence
  Laboratory synthesis
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGCGTKLEIK

SEQ ID NO: 6 - protein, 119 aa, Artificial Sequence
  Laboratory synthesis
QVQLQQSGAELARPGASVKMSCKASGYTFTRYTMHWVKQRPGQCLEWIGYINPSRGYTNYNQKFKDKATLTTDKSSSTAYMQLSSLTSEDSAVYYCARYYDDHYCLDYWGQGTTLTVSS

SEQ ID NO: 7 - DNA, 318 bp, Artificial Sequence
  Laboratory synthesis
cagatcgtgctgacacagagccccgccatcatgagtgctagccctggcgagaaagtgaccatgacctgtagcgccagcagcagcgtgtcctacatgaactggtatcagcagaagtccggcacaagccccaagcggtggatctacgatacaagcaagctggcctctggcgtgcccgctcactttagaggatctggcagcggcaccagctacagcctgacaatctctggcatggaagccgaggatgccgccacctactattgccagcagtggtccagcaatcccttcacctttggctgtggcaccaagctggaaatcaaa

SEQ ID NO: 8 - DNA, 357 bp, Artificial Sequence
  Laboratory synthesis
caggttcagctgcaacagtctggcgccgaacttgctagaccaggcgccagcgtgaagatgagctgtaaagccagcggctacaccttcaccagatacaccatgcactgggtcaagcagaggcccggacagtgccttgagtggatcggctacatcaaccccagccggggctacaccaactacaaccagaagttcaaggacaaggccacactgaccaccgacaagagcagcagcacagcctacatgcagctgagcagcctgaccagcgaagatagcgccgtgtactactgcgcccggtactacgacgatcactactgcctggattactggggccagggcacaaccctgacagtgtcatct

SEQ ID NO: 9 - protein, 107 aa, Artificial Sequence
  Laboratory synthesis
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGCGTKVEIK

SEQ ID NO: 10 - protein, 122 aa, Artificial Sequence
  Laboratory synthesis
EVQLVESGGGLVQPGGSLRLSCAASGYSFTGYTMNWVRQAPGKCLEWVALINPYKGVTTYADSVKGRFTISVDKSKNTAYLQMNSLRAEDTAVYYCARSGYYGDSDWYFDVWGQGTLVTVSS

SEQ ID NO: 11 - DNA, 321 bp, Artificial Sequence
  Laboratory synthesis
gatatccagatgacacagagccctagcagcctgtctgccagcgtgggagacagagtgaccatcacctgtagagccagccaggacatccggaactacctgaactggtatcagcagaagcccggcaaggcccctaagctgctgatctactacaccagcagactggaaagcggcgtgcccagcagattttctggcagcggcagcggaaccgactacaccctgaccatatctagcctgcagcctgaggacttcgccacctactattgccagcagggcaacaccctgccttggacctttggctgtggcaccaaggtggaaatcaaa

SEQ ID NO: 12 - DNA, 366 bp, Artificial Sequence
  Laboratory synthesis
gaagtgcagctggttgaatcaggcggaggcctggttcagccaggcggatctctgagactgtcttgtgccgcctccggctacagctttaccggctacaccatgaattgggtccgacaggcccctggcaagtgcctggaatgggttgccctgatcaacccctacaagggcgtgaccacatacgccgactctgtgaagggcagattcaccatcagcgtggacaagagcaagaacaccgcctacctgcagatgaacagcctgagagccgaggacaccgccgtgtattattgcgccagaagcggctactacggcgacagcgactggtactttgatgtgtggggacagggaaccctggtcaccgtgtctagc

SEQ ID NO: 13 - protein, 15 aa, Artificial Sequence
  Laboratory synthesis
GGGGSGGGGSGGGGS

SEQ ID NO: 14 - DNA, 735 bp, Artificial Sequence
  Laboratory synthesis
cagatcgtgctgacacagagccccgccatcatgagtgctagccctggcgagaaagtgaccatgacctgtagcgccagcagcagcgtgtcctacatgaactggtatcagcagaagtccggcacaagccccaagcggtggatctacgatacaagcaagctggcctctggcgtgcccgctcactttagaggatctggcagcggcaccagctacagcctgacaatctctggcatggaagccgaggatgccgccacctactattgccagcagtggtccagcaatcccttcacctttggctgtggcaccaagctggaaatcaaaggcggaggcggaagtggcggcggaggtagcggtggtggtggaagcggtggcggaggatctcaggttcagctgcaacagtctggcgccgaacttgctagaccaggcgccagcgtgaagatgagctgtaaagccagcggctacaccttcaccagatacaccatgcactgggtcaagcagaggcccggacagtgccttgagtggatcggctacatcaaccccagccggggctacaccaactacaaccagaagttcaaggacaaggccacactgaccaccgacaagagcagcagcacagcctacatgcagctgagcagcctgaccagcgaagatagcgccgtgtactactgcgcccggtactacgacgatcactactgcctggattactggggccagggcacaaccctgacagtgtcatct

SEQ ID NO: 15 - DNA, 732 bp, Artificial Sequence
  Laboratory synthesis
gatatccagatgacacagagccctagcagcctgtctgccagcgtgggagacagagtgaccatcacctgtagagccagccaggacatccggaactacctgaactggtatcagcagaagcccggcaaggcccctaagctgctgatctactacaccagcagactggaaagcggcgtgcccagcagattttctggcagcggcagcggaaccgactacaccctgaccatatctagcctgcagcctgaggacttcgccacctactattgccagcagggcaacaccctgccttggacctttggctgtggcaccaaggtggaaatcaaaggtggtggcggatctggtggtggtggaagtggcggtggcggttctgaagtgcagctggttgaatcaggcggaggcctggttcagccaggcggatctctgagactgtcttgtgccgcctccggctacagctttaccggctacaccatgaattgggtccgacaggcccctggcaagtgcctggaatgggttgccctgatcaacccctacaagggcgtgaccacatacgccgactctgtgaagggcagattcaccatcagcgtggacaagagcaagaacaccgcctacctgcagatgaacagcctgagagccgaggacaccgccgtgtattattgcgccagaagcggctactacggcgacagcgactggtactttgatgtgtggggacagggaaccctggtcaccgtgtctagc

SEQ ID NO: 16 - protein, 245 aa, Artificial Sequence
  Laboratory synthesis
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGCGTKLEIKGGGGSGGGGSGGGGSGGGGSQVQLQQSGAELARPGASVKMSCKASGYTFTRYTMHWVKQRPGQCLEWIGYINPSRGYTNYNQKFKDKATLTTDKSSSTAYMQLSSLTSEDSAVYYCARYYDDHYCLDYWGQGTTLTVSS

SEQ ID NO: 17 - protein, 244 aa, Artificial Sequence
  Laboratory synthesis
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGCGTKVEIKGGGGSGGGGSGGGGSEVQLVESGGGLVQPGGSLRLSCAASGYSFTGYTMNWVRQAPGKCLEWVALINPYKGVTTYADSVKGRFTISVDKSKNTAYLQMNSLRAEDTAVYYCARSGYYGDSDWYFDVWGQGTLVTVSS

SEQ ID NO: 18 - protein, 111 aa, Artificial Sequence
  Laboratory synthesis
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 19 - protein, 124 aa, Artificial Sequence
  Laboratory synthesis
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 20 - protein, 451 aa, Artificial Sequence
  Laboratory synthesis
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - DNA, 1353 bp, Artificial Sequence
  Laboratory synthesis
caagttcaacttcagcagtctggcgccgaactcgtgcggcctggatctagcgtgaagatcagctgtaaagccagcggctacgccttcagcagctactggatgaactgggtcaagcagaggcctggacagggcctcgaatggatcggacaaatttggcctggcgacggcgacaccaactacaacggcaagttcaagggcaaagccacactgaccgccgacgagtctagcagcacagcctacatgcagctgagcagcctggcctctgaagatagcgccgtgtacttctgcgccagacgggaaacaaccaccgtgggcagatattactacgccatggactactggggccagggcaccacagtgacagttagctctgcgtcgaccaagggccccagcgtgttccccctggccccttgcagcagaagcaccagcgagagcacagccgccctgggctgcctggtgaaggactacttccccgagcccgtgaccgtgtcctggaacagcggcgctctgaccagcggcgtgcataccttccccgccgtgctccagagcagcggactgtactccctgagcagcgtggtgacggtgccttccagcagcctgggcaccaagacctacacttgcaacgtggaccacaagcccagcaacaccaaggtggacaagagagtggagagcaagtacggccctccctgccccccttgccctgcccccgagttcctgggcggacctagcgtgttcctgttcccccccaagcccaaggacaccctgatgatcagcagaacccccgaggtgacgtgcgtggtggtggacgtgtcccaggaggaccccgaggtccagtttaattggtacgtggacggcgtggaagtgcataacgccaagaccaagcccagagaggagcagttcaacagcacctacagagtggtgtccgtgctgaccgtgctgcaccaggactggctgaacggcaaggaatacaagtgcaaggtctccaacaagggcctgcctagcagcatcgagaagaccatcagcaaggccaagggccagccacgggagccccaggtctacaccctgccacctagccaagaggagatgaccaagaaccaggtgtccctgacctgtctggtgaaaggcttctatcccagcgatatcgccgtggagtgggagagcaacggccagcccgagaacaactacaagaccaccccccctgtgctggacagcgacggcagcttcttcctgtactccagactgaccgtggacaagtccagatggcaggagggcaacgtcttcagctgctccgtgatgcacgaggccctgcacaaccactacacccagaagtccctgagcctgagcctgggcaag